(19) United States
(12) Patent Application Publication
(10) Pub. No.: US 2024/0041378 A1
(43) Pub. Date: Feb. 8, 2024

(54) Calibration of Positions of EEG Electrodes on a Subject

(71) Applicant: Telefonaktiebolaget LM Ericsson (publ), Stockholm (SE)

(72) Inventors: Pex Tufvesson, Lund (SE); Johan Eker, Lund (SE); Bo Bernhardsson, Lund (SE)

(21) Appl. No.: 18/257,473

(22) PCT Filed: Dec. 18, 2020
(86) PCT No.: PCT/EP2020/087183

§ 371 Date: Jun. 14, 2023

(51) Int. Cl. A61B 5/291; A61B 5/00

(52) U.S. Cl. CPC A61B 5/291; A61B 5/6803; A61B 5/684

(57) ABSTRACT

There is provided mechanisms for determining calibration of positions of EEG electrodes on a subject. A method is performed by a controller. The method comprises obtaining a set of signals representative of estimated positions for the EEG electrodes when placed on the subject. Each of the estimated positions represents a position of one of the EEG electrodes relative to the subject. The method comprises comparing the set of signals representative of estimated positions to a set of reference signals representative of reference positions. The set of reference positions represents intended positions for the EEG electrodes when placed on the subject. The method comprises determining the calibration of positions of the EEG electrodes using a difference between a property of the obtained signals and the set of reference signals.

### TECHNICAL FIELD

Embodiments presented herein relate to a method, a controller, a computer program, and a computer program product for determining calibration of positions of electroencephalography electrodes on a subject.

### BACKGROUND

Brain-computer interfaces (BCIs) is the general term for any technology that communicates directly with the brain, either to extract information from it, or to inject information into it by means of brain stimulation. The potential use cases are numerous and range from supporting paraplegics with spinal injuries to be able to move a cursor on the screen or even their limbs, to more futuristic scenarios with augmented cognition, for example providing external working memory.

State-of-the-art BCIs are based on electroencephalography (EEG), which is a non-invasive method to record the electrical activity of the brain with electrodes placed on the scalp. When placed on the skull of a human being, the EEG electrodes record activity in the subject's 86 billion neurons. Alternative methods for acquiring brain signals such as functional magnetic resonance imaging (fMRI), positron emission tomography (PET) and magnetoencephalography (MEG) are generally considered to be too bulky, too expensive and/or too cumbersome to be used for consumer grade everyday BCI. Invasive methods such as brain EEG electrode implants require surgically opening the skull, which exposes the subject to risks currently not worth taken except in exceptional situations.

BCI is a relatively new technology and current expectations are higher than the delivered performance of today's products. As an example, state-of-the-art in terms of machine learning (ML) for BCI using EEG is today hampered by sensitivity to noisy data. The issue is amplified by the fact that inter-subject, inter-session and inter-task variation in EEG data is large, and therefore extensive training is currently needed for good performance. Individual training of BCIs is a cumbersome and tedious process, hence only the dedicated and highly motivated subjects are successful in using today's BCI.

FIG. 1 schematically illustrates EEG electrodes 110 placed on a subject 100. As illustrated in FIG. 1, the EEG electrodes 110 need to be touching the outside of the skull of the subject 100. The EEG electrodes 110 are held in place using mechanical means. The mechanical means could be a cap, be part of a virtual reality head-mounted display, earpiece, sunglasses, tentacle-like support, or the like. As illustrated in FIG. 1, the EEG electrodes 110 are placed on the skull in a given pattern. Any improvement in the measurement recordings from the EEG electrodes 110 will improve the reliability of the BCI and make the subject's initial setup and calibration process for using a BCI shorter.

Research grade wired EEGs use hundreds of EEG electrodes 110, whereas consumer grade wireless systems use 2 to 24 EEG electrodes 110. To work properly, the EEG electrodes 110 require careful calibration. The pattern of the EEG electrodes 110 in FIG. 1 corresponds to the locations of the international 10-20 system for EEG recording. The precision of the measurements obtained from the EEG electrodes 110 is highly dependent of the exact location of the EEG electrodes 110 and requires tedious manual labor. Even when the EEG electrodes 110 are fitted to a cap at fixed places, this still requires the position of the cap to be carefully adjusted on the subject 100.

Hence, there is a need for simplified, but still accurate, fitting of EEG electrodes 110 on a subject.

### SUMMARY

An object of embodiments herein is to address the above issues by providing efficient calibration of the positions at which the EEG electrodes are placed on a subject.

According to a first aspect there is presented a method for determining calibration of positions of EEG electrodes on a subject. The method is performed by a controller. The method comprises obtaining a set of signals representative of estimated positions for the EEG electrodes when placed on the subject. Each of the estimated positions represents a position of one of the EEG electrodes relative the subject. The method comprises comparing the set of signals representative of estimated positions to a set of reference signals representative of reference positions. The set of reference positions represents intended positions for the EEG electrodes when placed on the subject. The method comprises determining the calibration of positions of the EEG electrodes using a difference between a property of the obtained signals and the set of reference signals.

According to a second aspect there is presented a controller for determining calibration of positions of EEG electrodes on a subject. The controller comprises processing circuitry. The processing circuitry is configured to cause the controller to obtain a set of signals representative of estimated positions for the EEG electrodes when placed on the subject. Each of the estimated positions represents a position of one of the EEG electrodes relative the subject. The processing circuitry is configured to cause the controller to compare the set of signals representative of estimated positions to a set of reference signals representative of reference positions. The set of reference positions represents intended positions for the EEG electrodes when placed on the subject. The processing circuitry is configured to cause the controller to determine the calibration of positions of the EEG electrodes using a difference between a property of the obtained signals and the set of reference signals.

According to a third aspect there is presented a controller for determining calibration of positions of EEG electrodes on a subject. The controller comprises an obtain module configured to obtain a set of signals representative of estimated positions for the EEG electrodes when placed on the subject. Each of the estimated positions represents a position of one of the EEG electrodes relative the subject. The controller comprises a compare module configured to compare the set of signals representative of estimated positions to a set of reference signals representative of reference positions. The set of reference positions represents intended positions for the EEG electrodes when placed on the subject. The controller comprises a determine module configured to determine the calibration of positions of the EEG electrodes using a difference between a property of the obtained signals and the set of reference signals.

According to a fourth aspect there is presented a computer program for determining calibration of positions of EEG electrodes on a subject, the computer program comprising computer program code which, when run on a controller, causes the controller to perform a method according to the first aspect.

According to a fifth aspect there is presented a computer program product comprising a computer program according to the fourth aspect and a computer readable storage medium on which the computer program is stored. The computer readable storage medium could be a non-transitory computer readable storage medium.

Advantageously, these aspects provide efficient calibration of the positions of the EEG electrodes.

Advantageously, these aspects enable efficient calibration of the positions at which the EEG electrodes are placed on the subject.

Advantageously, these aspects enable simple, but still accurate, fitting of the EEG electrodes on the subject.

Advantageously, knowing the physical location of the EEG electrodes on the subject will reduce the need for calibration at each new session, perhaps even completely removing the need for initial calibration at the start of each and every new session.

Advantageously, these aspects can simplify usage for any type of caps with EEG electrodes, even caps with less strict orientation and fit.

Other objectives, features and advantages of the enclosed embodiments will be apparent from the following detailed disclosure, from the attached dependent claims as well as from the drawings.

Generally, all terms used in the claims are to be interpreted according to their ordinary meaning in the technical field, unless explicitly defined otherwise herein. All references to “a/an/the element, apparatus, component, means, module, step, etc.” are to be interpreted openly as referring to at least one instance of the element, apparatus, component, means, module, step, etc., unless explicitly stated otherwise. The steps of any method disclosed herein do not have to be performed in the exact order disclosed, unless explicitly stated.

### BRIEF DESCRIPTION OF THE DRAWINGS

The inventive concept is now described, by way of example, with reference to the accompanying drawings, in which:

FIG. 1 schematically illustrates positions of EEG electrodes on a subject;

FIG. 2 schematically illustrates a subject wearing a cap on which EEG electrodes are attached according to an embodiment;

FIG. 3 is a flowchart of methods according to embodiments;

FIG. 4 and FIG. 5 schematically illustrate setups where EEG electrodes are placed on a subject according to embodiments

FIG. 6 is a schematic diagram showing functional units of a controller according to an embodiment;

FIG. 7 is a schematic diagram showing functional modules of a controller according to an embodiment; and

FIG. 8 shows one example of a computer program product comprising computer readable storage medium according to an embodiment.

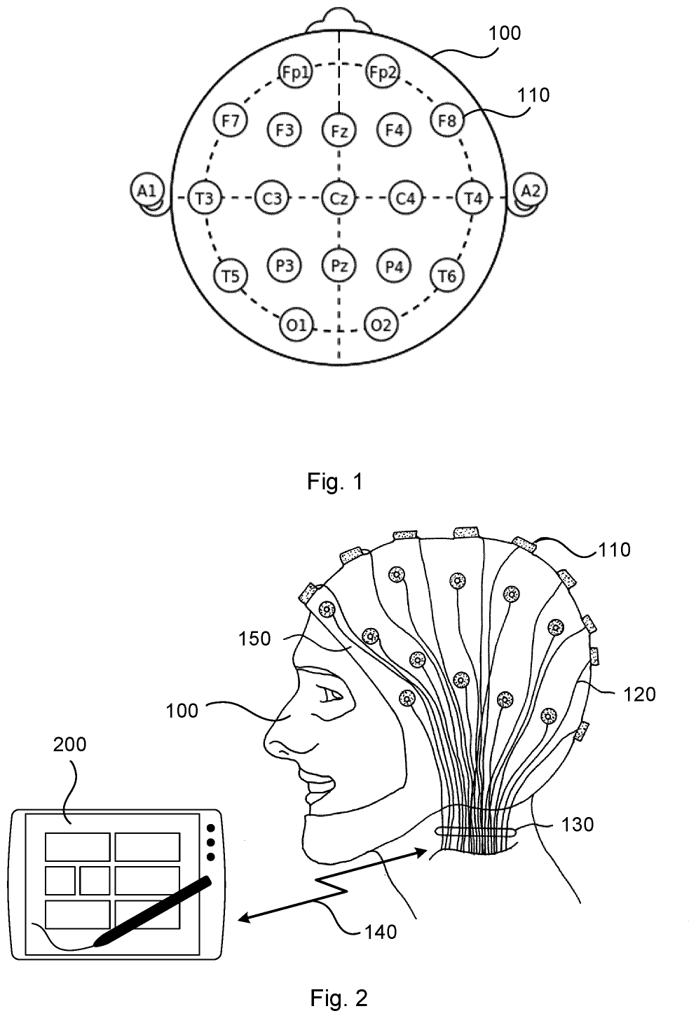

### DETAILED DESCRIPTION

The inventive concept will now be described more fully hereinafter with reference to the accompanying drawings, in which certain embodiments of the inventive concept are shown. This inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like elements throughout the description. Any step or feature illustrated by dashed lines should be regarded as optional.

As noted above there is a need for simplified, but still accurate, fitting of EEG electrodes 110 on a subject. In this respect, the physical placement of EEG electrodes 110 on the subject 100 is crucial for same-person new-session BCI performance. Even a small displacement of the EEG electrodes 110 impacts accuracy, and the tedious initial calibration and individual training needs to be redone prior to every session.

At least some of the herein disclosed embodiments enable an improved way of calibrating EEG electrode based BCI devices by measuring the positions of the EEG electrodes 110. The disclosed inventive concept will thereby decrease calibration time and thus improve usability of BCI devices with EEG electrodes 110.

The embodiments disclosed herein in particular relate to mechanisms for determining calibration of positions of EEG electrodes 110 on a subject 100. In order to obtain such mechanisms there is provided a controller 200, a method performed by the controller 200, a computer program product comprising code, for example in the form of a computer program, that when run on a controller 200, causes the controller 200 to perform the method.

FIG. 2 schematically illustrates a setup where EEG electrodes 110 are placed on the skull of a subject 100. The EEG electrodes 110 are attached to a cap 150 and are connected by wires 120 to a cable harness 130. The EEG electrodes 110 are in communication over a link 140 with a controller 200. The cable harness 130 might be provided with at least one transceiver for enabling communication between the EEG electrodes 110 and the controller 200, whereby the link 140 is wireless. Alternatively, the cable harness 130 is connected directly to the controller 200, whereby the link 140 is wired and defined by the cable harness 130 itself. In some examples, as in FIG. 2, the subject 100 is a human being (or an animal), and the EEG electrodes 110 are placed on the head, or skull, of the subject 100.

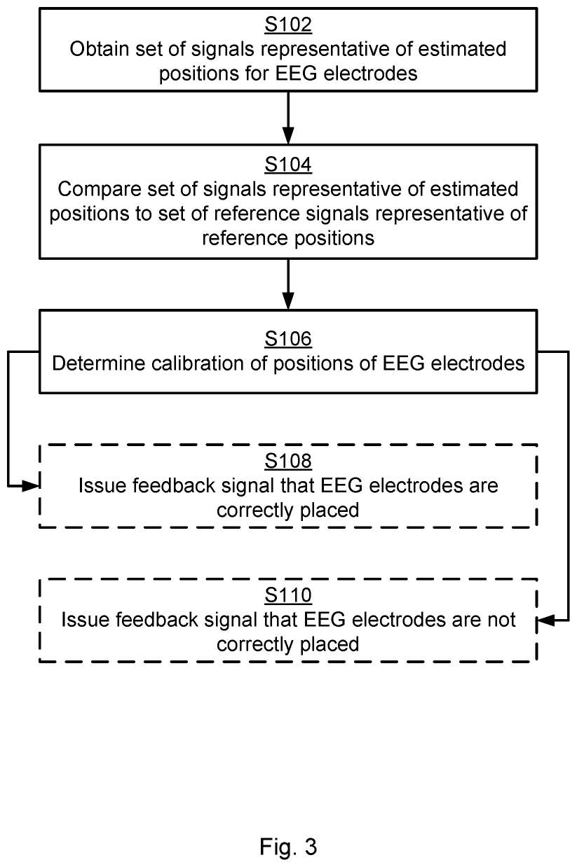

FIG. 3 is a flowchart illustrating embodiments of methods for determining calibration of positions of EEG electrodes 110 on a subject 100. The methods are performed by the controller 200. The methods are advantageously provided as computer programs 820.

Initially, EEG electrodes 110 are positioned on the subject 100. A test signal is transmitted from a known reference point and the test signal is picked up by the EEG electrodes 110 to create a map of the initial set-up of the EEG electrodes 110 in a training session. The training session may suitably be conducted versus a neural network containing a large amount of dynamic data from other subjects to make an initial calibration of the positions of the EEG electrodes 110. Alternatively, an educated guess of the positions of the EEG electrodes 110 are used. When the training session is concluded, data in terms of the positions of the EEG electrodes 110 on the subject 100 is stored.

When the EEG electrodes 110 are to again be positioned on the same subject 100, the EEG electrodes 110 are positioned in approximately the same positions. Calibration of the positions of the EEG electrodes 110 is then performed by applying the test signal at the same reference point as during the training session.

S102: The controller 200 obtains a set of signals representative of estimated positions (xⁱ, z^{j}) for the EEG electrodes 110 when placed on the subject 100. Each of the estimated positions represents a position of one of the EEG electrodes 110 relative to the subject 100.

Using previously stored data, the difference to a previous session(s), possible for a different user, can thus be found.

S104: The controller 200 compares the set of signals representative of estimated positions to a set of reference signals representative of reference positions (xⁱ*, z^{j}*). The set of reference positions represents intended positions for the EEG electrodes 110 when placed on the subject 100.

S106: The controller 200 determines the calibration of positions of the EEG electrodes 110 using a difference between a property of the obtained signals and the set of reference signals.

In some aspects, the property depends on the distance between the estimated positions and the intended positions. In some aspects, the property is expressed in terms of a time of arrival value, a time difference of arrival value, a phase value or an attenuation value.

There may be different actions taken by the controller 200 once having determined the calibration of positions of the EEG electrode 110 and before measurements are to be collected by the EEG electrodes 110 from the subject 100.

In some aspects, a (first) feedback signal is issued when the set of estimated positions of the EEG electrodes 110 is successfully calibrated. That is, in some embodiments, the controller 200 is configured to perform (optional) step S108:

S108: The controller 200 issues a (first) feedback signal representing that the EEG electrodes 110 are correctly placed on the subject 100.

In some aspects, the set of estimated positions of the EEG electrodes 110 are determined to be calibrated when a difference between the set of estimated positions and the set of reference positions is less than a threshold value. There could be different actions taken by the controller 200 when the difference is not less than the threshold value. The controller 200 might be configured to either compensate for the difference or issue a (second) feedback indicating that the positions of the EEG electrodes 110 need adjustment. In particular, in some embodiments, when the difference is not less than the threshold value, the difference is utilized as a compensation factor when the EEG electrodes 110 are in a state of operation. In some embodiments, the controller 200 is configured to perform (optional) step S110 when the difference is not less than the threshold value:

S110: The controller 200 issues a (second) feedback signal representing that the EEG electrodes 110 are not correctly placed on the subject 100.

The (second) feedback signal could act as a trigger for adjusting the positions of the EEG electrodes 110 on the subject 100. Step S110 could be performed when the difference is so large that the controller 200 is incapable of utilizing the difference as a compensation factor when the EEG electrodes 110 are in a state of operation

Embodiments relating to further details of determining calibration of positions of EEG electrodes 110 on a subject 100 as performed by the controller 200 will now be disclosed.

In some embodiments, there is one reference position for each of the estimated positions. In this case, each reference position could correspond to an intended position for one of the EEG electrodes 110. In other embodiments, one and the same reference position is shared between at least two estimated positions. The one and the same reference position might be shared between the at least two estimated positions by means of interpolation, or the like. This allows further EEG electrodes 110 to be added over time, without the need to perform a new initial calibration each time a new EEG electrode 110 is added. In similar way, there might be two or more reference positions for each estimated position. This allows for an initial calibration to be performed for more EEG electrodes 110 than used during a subsequent session.

In some aspects, the reference positions are obtained during previous calibration. That is, in some embodiments, the set of reference positions stem from previous determining of calibration of the positions of the EEG electrodes 110 on the same subject 100 or on another subject 100.

There could be different ways for the controller 200 to obtain the set of estimated positions for the EEG electrodes 110 when placed on the subject 100. In some embodiments, the signals representative of estimated positions are obtained from location measurements of the EEG electrodes 110. The location measurements might be made on a test signal as picked up the EEG electrodes 110 upon the test signal having been transmitted from at least one position. In further detail, the distances between the EEG electrodes 110, and thereby their positions, might be estimated by transmission of the test signals from known locations and the measure the time difference of arrival of the test signal at the EEG electrodes 110. That is, in some embodiments, the location measurements pertain to time of arrival values or time difference of arrival values of the test signal at the EEG electrodes 110. Time of arrival values might be used when the timing of the test signal is known, whereas time difference of arrival values might be used when the timing of the test signal is unknown. The test signals will thus propagate across the surface of the subject 100 (such as across the skin of the skull) with a certain speed. Since the separate EEG electrodes 110 are wired, time difference of arrival of anomalies can be used for both Riemannian electrode manifold localization as well as Carthesian relative xyz localization of the positions of the EEG electrodes 110. A small electrical impulse will distribute across the skull and having a few of them initiated from different locations together with time difference of arrival values determined at each of the EEG electrodes 110 enables precise localization of the EEG electrodes 110.

Time-delay estimation using known test signals (comprising one or more test symbols) might therefore be performed to find the positions of the EEG electrodes 110. The positions are obtained from distance measurements between the signal emitter of the test signal and the signal receiver of the test signal.

In general terms, assuming that the received signal r(t) at the signal receiver is a delayed and attenuated version of the transmitted signal s(t) at the signal emitter, the received signal r(t) can be expressed as follows:

r(t)=a·s(t−τ)+e(t),

where T is the time delay proportional to the distance d between signal emitter and signal receiver, where a is an attenuation factor, and where e(t) represents noise. In this respect, any filtering of the test signal at the signal receiver to identify the reception of the one or more test symbols can be done as part of the BCI signal processing chain using technologies that as such are known in the art. Time delay estimation can be performed by correlating time shifted versions of the received signal r(t) with the transmitted signal s(t) and, where the time delay yields highest correlation. That is:

τ=arg max∫r(t)s(t−τ)dt.

The time delay τ is then transformed to a distance measurement d as follows:

$$d = \frac{τ}{v} ,$$

where v is the propagation speed of the test signal. Hence, in some embodiments, each of the time difference of arrival values is representative of a distance, denoted d^{ij}, between two of the EEG electrodes 110.

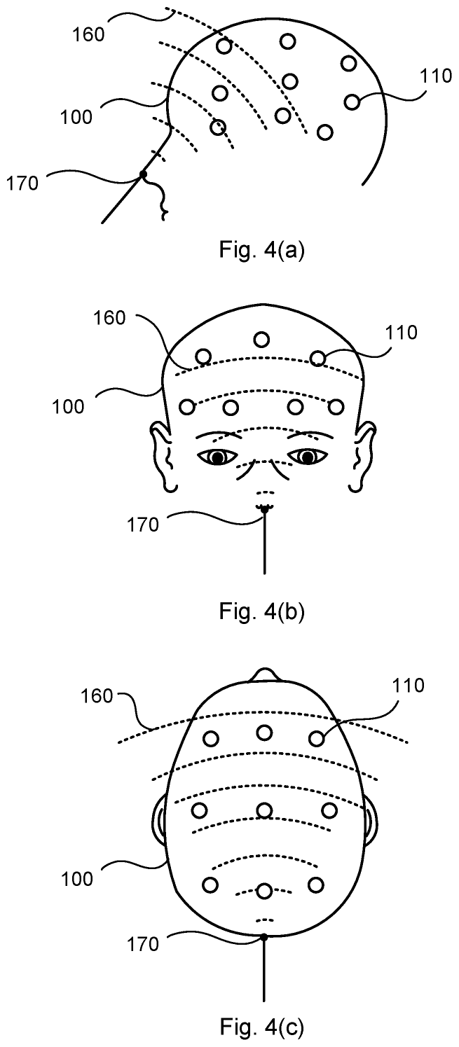

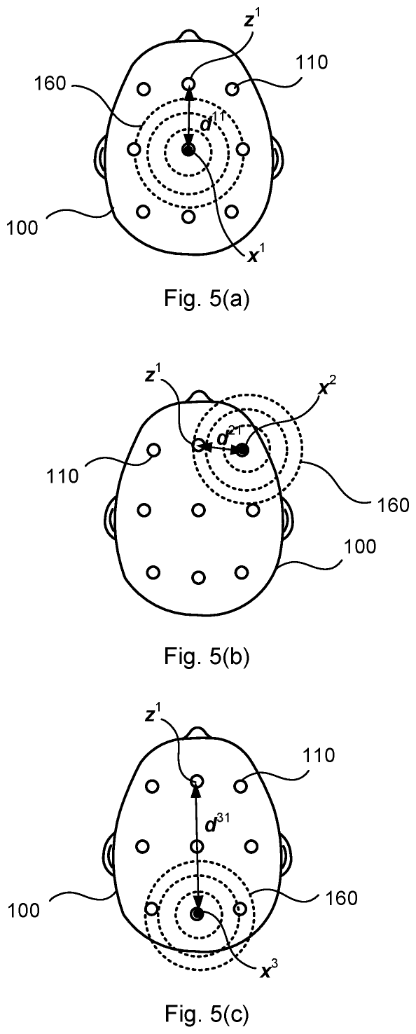

Let xⁱ=(x₁ⁱ, x₂ⁱ, x₃ⁱ) denote a vector with the 3D coordinates of an electrode i emitting a test signal, where i=1 . . . M. Similarly, let z^{j}=(z₁^{j}, z₂^{j}, z₃^{j}) denote the 3D position of EEG electrode j receiving the test signal, where j=1 . . . N. Either electrode i is an external electrode (as in FIG. 4 which will be described below) or is one of the EEG electrodes 110 (as in FIG. 5 which will be described below). The vector d^{ij} then represents the distance between the position of electrode i and the position of EEG electrode j as obtained from the location measurements of the EEG electrodes 110. The distance measurement obtained from the correlation technique described above will then give a measurement equation in the form:

∥xⁱ−z^{j}∥=d^{ij}+e,

where e is a noise vector. This process can be repeated until the position of all the EEG electrodes 110 can be determined with sufficient precision. In general terms, the exact position of all EEG electrodes 110 can be determined using test signals transmitted from two or more locations. Hence, in some embodiments, there are at least two positions with known relation relative the subject 100.

In general terms, the test signal is known in terms of the shape the pulse of the test signal has when being transmitted. In further aspects, there could be different positions and sources from which the test signal is transmitted.

As disclosed above, the test signal is transmitted from at least one position.

In some aspects, the test signal is an externally injected signal. That is, in some embodiments, the test signal is an externally generated signal transmitted from an electrode at this at least one position other that the EEG electrodes 110. In other aspects, one or more of the EEG electrodes 110 themselves are utilized for the transmission of the test signal. That is, in some embodiments, the test signal is generated by, and transmitted from, at least one of the EEG electrodes 110 at this at least one position.

In some embodiments, each of the at least one position (from which the test signal is sent) has a known relation relative the subject 100. This could be realized by transmitting an externally generated test signal from electrodes positioned at easily identifiable, and recognized positions of the subject 100 or by placing EEG electrodes 110 from which the test signal is to be transmitted at such easily identifiable, and recognized positions of the subject 100. Non-limiting examples of such easily identifiable, and recognized positions of the subject 100 are: the tip of the nose, the earlobes, the tip of the jaw.

A first illustrative example where the test signal is an externally generated signal is shown in FIG. 4. FIG. 4 illustrates at (a) a side view, at (b) a front view, and at (c) a top view of a setup where EEG electrodes 110 are placed on a subject 100. Dashed lines 160 show the propagation of the test signal over the subject 100. FIG. 4 could represent a possible setup where the subject 100 actively triggers a test signal and the controller 200 calculates the positions of the EEG electrodes 110 based on the reception of the test signal at the EEG electrodes 110. In FIG. 4 an external electrode 170 is placed on the nose (as in FIG. 4(a) and FIG. 4(b)) and at the back-off the head (as in FIG. 4(c)). These are examples of positions considered to be easy to identify for a user. Other easy to use positions for external electrodes 170 can be the ears. FIG. 4 represents an embodiment where the test signal is an externally generated signal transmitted from an electrode 170 at at least one position other than the EEG electrodes 110.

A second illustrative example where at least some of the EEG electrodes 110 are capable of emitting test signals is shown in FIG. 5. FIG. 5 illustrates at (a), (b), and (c) reception of a test signal at an EEG electrode positioned at the 3D coordinates z¹, and where the test signal is transmitted from an EEG electrode positioned at the 3D coordinates x¹, x², and x³, respectively, thus yielding distance vectors d¹¹, d²¹, and d³¹, respectively. Dashed lines 160 show the propagation of the test signal over the subject 100. FIG. 5 represents an embodiment where the test signal is generated by, and transmitted from, at least one of the EEG electrodes 110.

Such measurements as performed between M signal emitters with 3D position vectors x¹, . . . , x^{M} and N receiving EEG electrodes with 3D position vectors z¹, . . . , z^{N} yields a system of M·N nonlinear equations. The positions of all 3D position vectors x¹, . . . , x^{M} and z¹, . . . , z^{N} can then be obtained by solving a nonlinear least squares problem. In some aspects, the nonlinear least squares problem pertain to minimization of the difference between a) distances between pairs of EEG electrodes 110 obtained from measurements where one of the electrodes has a known position relative to the subject 100, and b) the corresponding distances between known positions and estimated positions. In particular, in some embodiments, the set of estimated positions is obtained by solving a nonlinear least squares problem that pertains to minimization of a deviation between first distances (defined by the factor ∥xⁱ−z^{j}∥) and second distances (defined by the factor d^{ij}). The first distances are estimated distances between the estimated positions of the EEG electrodes 110 and the second distances are the distances obtained from the location measurements of the EEG electrodes 110. In particular, in some embodiments, the nonlinear least squares problem involves finding vectors x¹, . . . , x^{M}, and vectors z¹, . . . , z^{N} that minimize:

$${\sum }_{i = 1}^{M} {\sum }_{j = 1}^{N} abs ( ‖ {x}^{i} - {z}^{i} ‖ - {d}^{ij} ) ,$$

where vector xⁱ thus represents the position of electrode i, where i=1 . . . M, from which the test signal is transmitted, where vector z^{j} represents the position of EEG electrode j, where j=1 . . . N, at which the test signal is received from electrode i. As noted above, either electrode i is an external electrode 170 (as in FIG. 4) or is one of the EEG electrodes 110 (as in FIG. 5).

In some aspects, initial guesses of the positions of the EEG electrodes 110 are used as input to the algorithm used to solve the nonlinear least squares problem. This is a reasonable assumption since the EEG electrodes 110 are not randomly placed on the subject 100; there might generally be a usable known relative order between the EEG electrodes 110. Hence, in some embodiments, the nonlinear least squares problem is solved using a gradient descent technique with an initial estimate of the positions for the EEG electrodes 110 when placed on the subject 100 as constraints.

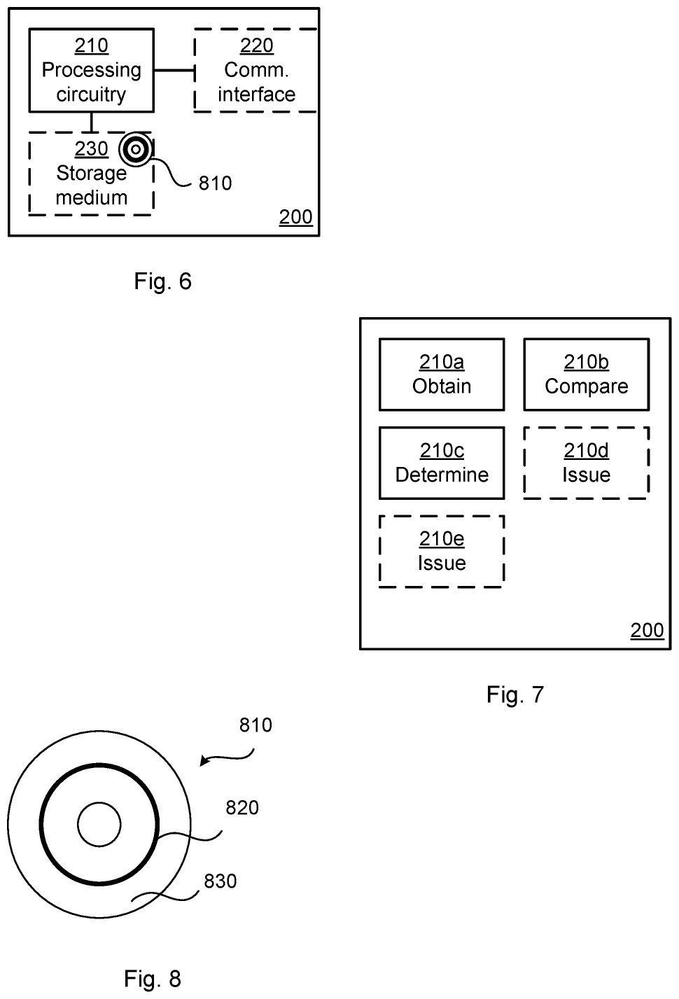

FIG. 6 schematically illustrates, in terms of a number of functional units, the components of a controller 200 according to an embodiment. Processing circuitry 210 is provided using any combination of one or more of a suitable central processing unit (CPU), multiprocessor, microcontroller, digital signal processor (DSP), etc., capable of executing software instructions stored in a computer program product 810 (as in FIG. 8), e.g. in the form of a storage medium 230. The processing circuitry 210 may further be provided as at least one application specific integrated circuit (ASIC), or field programmable gate array (FPGA).

Particularly, the processing circuitry 210 is configured to cause the controller 200 to perform a set of operations, or steps, as disclosed above. For example, the storage medium 230 may store the set of operations, and the processing circuitry 210 may be configured to retrieve the set of operations from the storage medium 230 to cause the controller 200 to perform the set of operations. The set of operations may be provided as a set of executable instructions.

Thus the processing circuitry 210 is thereby arranged to execute methods as herein disclosed. The storage medium 230 may also comprise persistent storage, which, for example, can be any single one or combination of magnetic memory, optical memory, solid state memory or even remotely mounted memory. The controller 200 may further comprise a communications interface 220 at least configured for communications with other entities, functions, nodes, and devices, such as the EEG electrodes 110 via at least one transceiver in the cable harness 130 over the link 140. As such the communications interface 220 may comprise one or more transmitters and receivers, comprising analogue and digital components. The processing circuitry 210 controls the general operation of the controller 200 e.g. by sending data and control signals to the communications interface 220 and the storage medium 230, by receiving data and reports from the communications interface 220, and by retrieving data and instructions from the storage medium 230. Other components, as well as the related functionality, of the controller 200 are omitted in order not to obscure the concepts presented herein.

FIG. 7 schematically illustrates, in terms of a number of functional modules, the components of a controller 200 according to an embodiment. The controller 200 of FIG. 7 comprises a number of functional modules; an obtain module 210a configured to perform step S102, a compare module 210b configured to perform step S104, and a determine module 210c configured to perform step S106. The controller 200 of FIG. 7 may further comprise a number of optional functional modules, such as any of a first issue module 210d configured to perform step S108, and a second issue module 210e configured to perform step S108. In general terms, each functional module 210a:210e may in one embodiment be implemented only in hardware and in another embodiment with the help of software, i.e., the latter embodiment having computer program instructions stored on the storage medium 230 which when run on the processing circuitry makes the controller 200 perform the corresponding steps mentioned above in conjunction with FIG. 7. It should also be mentioned that even though the modules correspond to parts of a computer program, they do not need to be separate modules therein, but the way in which they are implemented in software is dependent on the programming language used. Preferably, one or more or all functional modules 210a:210e may be implemented by the processing circuitry 210, possibly in cooperation with the communications interface 220 and/or the storage medium 230. The processing circuitry 210 may thus be configured to from the storage medium 230 fetch instructions as provided by a functional module 210a:210e and to execute these instructions, thereby performing any steps as disclosed herein.

The controller 200 may be provided as a standalone device or as a part of at least one further device. Alternatively, functionality of the controller 200 may be distributed between at least two devices, or nodes. Thus, a first portion of the instructions performed by the controller 200 may be executed in a first device, and a second portion of the of the instructions performed by the controller 200 may be executed in a second device; the herein disclosed embodiments are not limited to any particular number of devices on which the instructions performed by the controller 200 may be executed. Hence, the methods according to the herein disclosed embodiments are suitable to be performed by a controller 200 residing in a cloud computational environment. Therefore, although a single processing circuitry 210 is illustrated in FIG. 6 the processing circuitry 210 may be distributed among a plurality of devices, or nodes. The same applies to the functional modules 210a:210e of FIG. 7 and the computer program 820 of FIG. 8.

The inventive concept has mainly been described above with reference to a few embodiments. However, as is readily appreciated by a person skilled in the art, other embodiments than the ones disclosed above are equally possible within the scope of the inventive concept, as defined by the appended patent claims.

1-40. (canceled)

41. A method for determining calibration of positions of electroencephalography (EEG) electrodes on a subject, the method being performed by a controller, the method comprising:
obtaining a set of signals representative of estimated positions (xⁱ, z^{j}) for the EEG electrodes when placed on the subject, wherein each of the estimated positions represents a position of one of the EEG electrodes relative to the subject;
comparing the set of signals representative of estimated positions to a set of reference signals representative of reference positions (xⁱ*, z^{j}*), wherein the set of reference positions represents intended positions for the EEG electrodes when placed on the subject; and
determining the calibration of positions of the EEG electrodes using a difference between a property of the obtained signals and the set of reference signals.

42. The method of claim 41, wherein the signals representative of estimated positions are obtained from location measurements of the EEG electrodes.

43. The method of claim 42, wherein the location measurements are made on a test signal as picked up the EEG electrodes upon the test signal having been transmitted from at least one position.

44. The method of claim 43, wherein the test signal is an externally generated signal transmitted from an electrode at said at least one position other than that of the EEG electrodes.

45. The method of claim 43, wherein the test signal is generated by, and transmitted from, at least one of the EEG electrodes at said at least one position.

46. The method of claim 43, wherein each of the at least one position has a known relation relative to the subject.

47. The method of claim 46, wherein there are at least two positions with known relation relative the subject.

48. The method of claim 42, wherein the location measurements pertain to time of arrival values or time difference of arrival values of the test signal at the EEG electrodes.

49. The method of claim 48, wherein each of the time of arrival values or time difference of arrival values is representative of a distance (di) between two of the EEG electrodes.

50. The method of claim 49, wherein the set of estimated positions is obtained by solving a nonlinear least squares problem pertaining to minimization of a deviation between first distances and second distances, where the first distances are estimated distances between the estimated positions of the EEG electrodes and the second distances are the distances obtained from the location measurements of the EEG electrodes.

51. The method of claim 50, wherein the nonlinear least squares problem involves finding vectors x¹, . . . , x^{M}, and vectors z¹, . . . , z^{N} that minimize:
$${\sum }_{i = 1}^{M} {\sum }_{j = 1}^{N} abs ( ‖ {x}^{i} - {z}^{i} ‖ - {d}^{ij} ) ,$$
where vector xⁱ represents the position of EEG electrode i, where i=1 . . . M, from which the test signal is transmitted, where vector z^{j} represents the position of EEG electrode j, where j=1 . . . N, at which the test signal is received from EEG electrode i, and where vector d^{ij} represents the distance between the position of EEG electrode i and the position of EEG electrode j as obtained from the location measurements of the EEG electrodes.

52. The method of claim 50, wherein the nonlinear least squares problem is solved using a gradient descent technique with an initial estimate of the positions for the EEG electrodes when placed on the subject as constraints.

53. The method of claim 41, wherein the method further comprises:
issuing a feedback signal representing that the EEG electrodes are correctly placed on the subject.

54. The method of claim 41, wherein, in response to the difference being not less than a threshold value, the difference is utilized as a compensation factor when the EEG electrodes are in a state of operation.

55. The method of claim 41, wherein the method further comprises:
issuing a feedback signal representing that the EEG electrodes are not correctly placed on the subject, in response to the difference being not less than a threshold value,

56. A controller for determining calibration of positions of electroencephalography (EEG) electrodes on a subject, the controller comprising processing circuitry, the processing circuitry being configured to cause the controller to:
obtain a set of signals representative of estimated positions (xⁱ, z^{j}) for the EEG electrodes when placed on the subject, wherein each of the estimated positions represents a position of one of the EEG electrodes relative to the subject;
compare the set of signals representative of estimated positions to a set of reference signals representative of reference positions (xⁱ*, z^{j}*), wherein the set of reference positions represents intended positions for the EEG electrodes when placed on the subject; and
determine the calibration of positions of the EEG electrodes using a difference between a property of the obtained signals and the set of reference signals.

57. The controller of claim 56, wherein the signals representative of estimated positions are obtained from location measurements of the EEG electrodes.

58. The controller of claim 57, wherein the location measurements are made on a test signal as picked up the EEG electrodes upon the test signal having been transmitted from at least one position.

59. The controller of claim 58, wherein the test signal is an externally generated signal transmitted from an electrode at said at least one position other than that of the EEG electrodes.

60. The controller of claim 58, wherein the test signal is generated by, and transmitted from, at least one of the EEG electrodes at said at least one position.

61. The controller of claim 58, wherein each of the at least one position has a known relation relative the subject.

62. The controller of claim 61, wherein there are at least two positions with known relation relative the subject.

63. The controller of claim 57, wherein the location measurements pertain to time of arrival values or time difference of arrival values of the test signal at the EEG electrodes.

64. The controller of claim 63, wherein each of the time of arrival values or time difference of arrival values is representative of a distance (di) between two of the EEG electrodes.

65. The controller of claim 64, wherein the set of estimated positions is obtained by solving a nonlinear least squares problem pertaining to minimization of a deviation between first distances and second distances, where the first distances are estimated distances between the estimated positions of the EEG electrodes and the second distances are the distances obtained from the location measurements of the EEG electrodes.
